(19) United States
(12) Patent Application Publication
(10) Pub. No.: US 2026/0021104 A1
(43) Pub. Date: Jan. 22, 2026

(54) BRILAROXAZINE LIPOSOME COMPOSITION

(71) Applicant: Reviva Pharmaceuticals, Inc., Cupertino, CA (US)

(72) Inventors: Laxminarayan Bhat, Cupertino, CA (US); Seema Rani Bhat, Cupertino, CA (US)

(21) Appl. No.: 19/340,389

(22) Filed: Sep. 25, 2025

Related U.S. Application Data
(63) Continuation of application No. PCT/US2024/022067, filed on Mar. 28, 2024.
(60) Provisional application No. 63/493,233, filed on Mar. 30, 2023.

(51) Int. Cl. A61K 31/538; A61K 9/127; A61K 47/10; A61K 47/28; A61K 47/32; A61K 47/36

(52) U.S. Cl. CPC A61K 31/538; A61K 9/127; A61K 47/10; A61K 47/28; A61K 47/32; A61K 47/36

(57) ABSTRACT

The present invention is directed to a liposome composition comprising brilaroxazine incorporated in bilayer lipid vesicles. In one embodiment, the liposome composition comprises bilayer lipid vesicles encapsulating an aqueous solution, wherein the bilayer lipid vesicles comprise one or more phospholipids, sterol, and brilaroxazine, and the aqueous solution comprises maltodextrin. The present invention is also directed to a pharmaceutical composition comprising the brilaroxazine liposome composition and a pharmaceutically acceptable carrier.

### CROSS-REFERENCE TO RELATED APPLICATION(S)

This application is a continuation of PCT/US2024/022067, filed Mar. 28, 2024; which claims the benefit of U.S. Provisional Application No. 63/493,233, filed Mar. 30, 2023. The contents of the above-identified applications are incorporated herein by reference in their entirety.

### FIELD OF THE INVENTION

The present invention relates to liposome composition comprising brilaroxazine, or a pharmaceutically acceptable salt therefore. The liposome composition comprising bilayer lipid vesicles encapsulating an aqueous solution, wherein the bilayer lipid vesicles comprise brilaroxazine, and the aqueous solution comprises maltodextrin.

### BACKGROUND

Liposomes are micro-particulate or colloidal carrier systems, usually 0.025-5.0 μm in diameter. Liposomes are composed of biodegradable, biocompatible components and provides a unique opportunity to deliver pharmaceuticals into the cells or even inside individual cellular compartments. Liposomes form spontaneously when the lipids are hydrated in aqueous media at the transition temperature. Lipids are composed of natural and/or synthetic lipids (phospholipids and sphingolipids) and may contain other bilayer constituents such as cholesterol and hydrophilic polymer lipids. FIG. 1 shows a representation a general structure of liposomes.

The compositions of liposomes determine interaction with blood and tissues. The compositions decide liposome's net physicochemical properties, namely membrane fluidity, charge density, and steric hindrance permeability. They are found to be useful carriers for both hydrophilic and hydrophobic drugs. These drug delivery systems employed for the delivery of drugs with varying lipophilicities, like water-soluble drug will be encapsulated within the aqueous compartment; the lipophilic drug is usually bound to the lipid bi-layer or dissolved in the lipid phase.

Brilaroxazine (RP5063) is a multimodal dopamine and 5-HT receptor modulator. Brilaroxazine displays a high binding affinity to D2-4 and 5-HT1A receptors as partial agonists, 5-HT2A as a weak partial agonist or neutral antagonist, 5-HT2B/7 as an antagonist, and a moderate affinity to serotonin transporter (SERT). Brilaroxazine has an established efficacy, safety, and pharmacokinetic profile from phase 1 and 2 studies in healthy volunteers and schizophrenia patients. Also, preclinical work indicates that this agent inhibits the release of multiple proinflammatory cytokines.

### BRIEF DESCRIPTION OF THE DRAWINGS

FIG. 1 shows a diagram of a general structure of a liposome.

FIG. 2 shows particles size (Z-average) and particles distribution index in the liposomal dispersion by DLS measurement of brilaroxazine liposomes.

FIG. 3 shows the HPLC chromatogram of brilaroxazine liposomes.

FIG. 4 shows the HPLC chromatogram of a lipogel sample. The first peak is brilaroxazine and the second peak is excipient.

FIG. 5 shows in-vitro diffusion study graph of lipogel through the membrane.

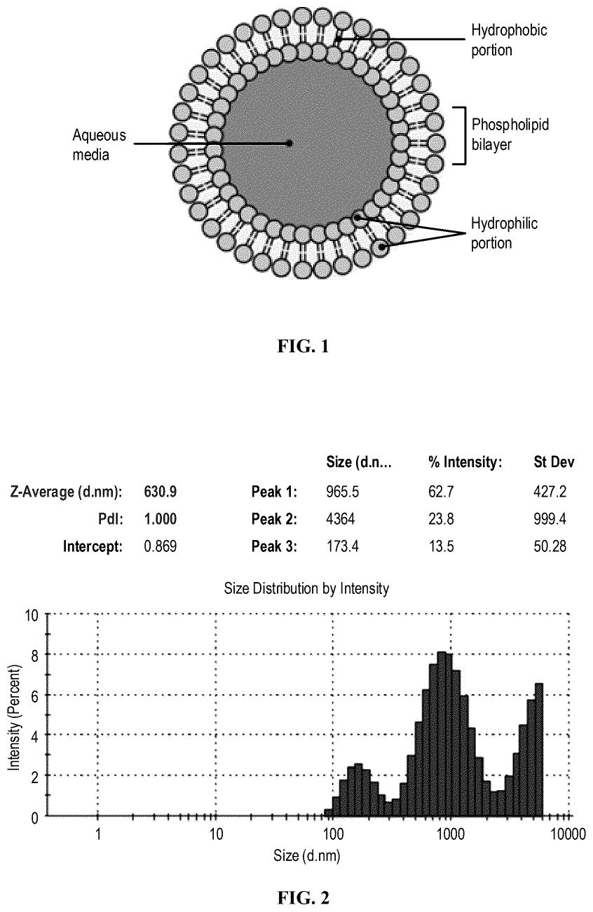

### DETAILED DESCRIPTION OF THE INVENTION

### Brilaroxazine Liposomes

The present invention is directed to a liposome composition comprising brilaroxazine incorporated in bilayer lipid vesicles. In one embodiment, the liposome composition comprises bilayer lipid vesicles encapsulating an aqueous solution, wherein the bilayer lipid vesicles comprise one or more phospholipids, sterol, and brilaroxazine, and the aqueous solution comprises maltodextrin.

Brilaroxazine (free based) is a basic and lipophilic molecule and has a molecular weight of 450.36 g/mol. Its chemical structure is shown below.

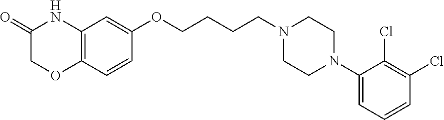 [SMILES: O=C1COc2ccc(OCCCCN3CCN(c4cccc(Cl)c4Cl)CC3)cc2N1]

Brilaroxazine often is the HCl salt form with a molecular weight of 486.7 g/mol.

Maltodextrin consists of D-glucose units connected in chains of variable length. The glucose units are primarily linked with α(1→4) glycosidic bonds. Maltodextrin is typically composed of a mixture of chains that vary from three to 17 glucose units long. Brilaroxazine liposomes encapsulating maltodextrin may provide a better drug release profile than that of brilaroxazine liposomes without maltodextrin.

The lipids used in forming the lipid vesicles typically include lipid mixtures composed predominantly of phospholipid(s) and sterol(s). A list of phospholipids used commonly in liposome preparations can be found on page 471 of Szoka et al (Ann Rev Biophys Bioeng (1980) 9:467). The vesicles may be formulated to include negatively or positively charged lipids, such as phosphatidic acid (PA) and phosphatidylglycerol (PG), to provide a desired surface charge on the reagent vesicles. A small amount of anti-oxidant such as α-tocopherol (0.1 to 1 mol %) can be added to the lipid mixture to increase the stability. A typical lipid mixture used in forming brilaroxazine liposome of the present invention includes phosphatidylcholine, cholesterol, and brilaroxazine.

The brilaroxazine liposomes are prepared by first dissolving the vesicle-forming lipids (e.g., brilaroxazine, phosphatidylcholine, cholesterol) in an inert organic solvent or solvent system, for example, chloroform and/or ethanol, to form an organic-phase solution of the lipids. In general, the inert organic solvent or solvent system is one in which the lipid components can be readily dissolved, at the concentration in the range of between about 0.5-50 mg lipid/mL. Then the lipid solution is dried completely to remove the organic solvent(s) and forms a thin lipid film on the surface of a vessel. For example, the lipid solution can be dried at 45-50° C. in a rotary evaporator using the vacuum to remove the solvent. Removal of all solvent leads to a thin film in the round bottom flask, which then remains in a vacuum for 12-24 hours to remove the traces in the thin lipid film completely. After drying, the thin lipid film is then hydrated with an aqueous solution. In a preferred embodiment, the aqueous solution contains maltodextrin.

In one embodiment, the brilaroxazine liposomes comprise 10-40% or 20-30% by weight of brilaroxazine.

In one embodiment, the brilaroxazine liposomes comprises 20-60% or 30-45% by weight of maltodextrin.

In one embodiment, the average particle size of the brilaroxazine liposomes is between 500-750 nm.

In one embodiment, the most intense peak of the brilaroxazine liposomes has a peak size between 900-1000 nM.

### Pharmaceutical Compositions

The present invention provides pharmaceutical compositions comprising one or more pharmaceutically acceptable carriers and brilaroxazine liposomes of the present invention. Brilaroxazine or its pharmaceutically acceptable salt in the pharmaceutical compositions in general is in an amount of about 0.01-20%, or 0.05-20%, or 0.1-20%, or 0.1-10%, or 0.1-5%, or 0.2-15%, or 0.2-10%, or 0.2-5%, or 0.2-2%, or 1-5% (w/w) for a topical formulation; about 0.1-5% for an injectable formulation, 0.1-5% for a patch formulation, about 1-90% for a tablet formulation, and 1-100% for a capsule formulation.

In one embodiment, brilaroxazine liposome is incorporated into any acceptable carrier, including creams, gels, lotions or other types of suspensions that can stabilize the active compound and deliver it to the affected area by topical applications. In another embodiment, the pharmaceutical composition can be in a dosage form such as tablets, capsules, granules, fine granules, powders, syrups, suppositories, injectable solutions, patches, or the like. The above pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Other pharmaceutically acceptable carriers include xanthan gum, carrageenan, Avicel RC-591 (a combination of microcrystalline cellulose and), and polyethylene glycol. Alternately, the active compound may be dissolved or suspended in a pharmaceutically acceptable lipid formulation such as those described by Kalepu et al (Acta Pharmaceutica Sinica B, 3: 361-372, 2013), for example, vegetable oil, coconut oil, castor oil, etc.

Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the brilaroxazine liposome may contain other excipients that have no bioactivity and no reaction with the active compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of excipients of a tablet or a capsule include, but not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone. For example, a tablet formulation may contain inactive ingredients such as colloidal silicon dioxide, crospovidone, hypromellose, magnesium stearate, microcrystalline cellulose, polyethylene glycol, sodium starch glycolate, and/or titanium dioxide. A capsule formulation may contain inactive ingredients such as gelatin, magnesium stearate, and/or titanium dioxide.

For example, a patch formulation of the active compound may comprise some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters or diethylene glycol monoethyl ether.

Topical formulations including the active compound can be in a form of gel, cream, lotion, liquid, emulsion, ointment, spray, solution, and suspension. The inactive ingredients in the topical formulations for example include, but not limited to, (emollient/permeation enhancer), diethylene glycol monoethyl ether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent).

The present application further provides a gel formulation comprising the brilaroxazine liposome composition, a gelling agent and a humectant. In one embodiment, the gel formulation comprises brilaroxazine in an amount of 0.005-10%, 0.01 to 5%, or 0.1-2% by weight. In one embodiment, the formulation has a gel appearance, and the bilayer lipid vesicles are intact and stable in the gel. In one embodiment, the gelling agent is carbomer 940, and the humectant is glycerin.

### Method of Use

The present application provides a method for treating psoriasis. The method comprises administering an effective amount of brilaroxazine liposomes to a subject in need thereof. “An effective amount,” as used herein, is the amount effective to treat psoriasis by ameliorating the pathological condition or reducing the symptoms of psoriasis. The method reduces the one or more signs and symptoms selected from the group consisting of: red patches of skin covered with thick and silvery scales; small scaling spots; dry and cracked skin; itching, burning, or soreness of the skin; thickened, pitted, or ridged nails, and swollen and stiff joints.

The pharmaceutical composition of the present invention can be applied by local administration and systemic administration. Local administration includes topical administration. Commonly used conventional semisolid dosage forms have certain limitations in drug delivery due to barrier properties of the skin. The skin is continuously involved in the construction of an efficient homeostatic barrier.

The brilaroxazine liposomes gel formulation provides the following advantages. Liposomes are microscopic vesicles that contain amphipathic phospholipids arranged in one or more concentric bilayers enclosing aqueous compartments. As a spherical shell, liposomes resemble biological membranes. Liposomes are composed of biodegradable, biocompatible components and provide a drug delivery system to deliver pharmaceuticals into the cells or even inside individual cellular compartments. Hence, the brilaroxazine liposomes gel formulation may deliver brilaroxazine in the deeper layer of the skin in severe psoriatic conditions.

Systemic administration includes oral, parenteral (such as intravenous, intramuscular, subcutaneous or rectal), and other systemic routes of administration. In systemic administration, the active compound first reaches plasma and then distributes into target tissues. Topical administration and oral administration are preferred routes of administration for the present invention.

In one embodiment, the composition is applied topically onto the affected area and rubbed into it. The composition is topically applied at least 1 or 2 times a day, or 3 to 4 times per day, depending on the medical issue and the disease pathology being chronic or acute. In general, the topical composition comprises about 0.01-10% (w/w) of the active compound. For example, the topical composition comprises about 0.1 to 2% (w/w) of the active compound. Depending on the size of the affected area, 0.2-85 mL, typically 0.2-10 mL, of the topical composition is applied to the individual per dose. The active compound passes through skin and is delivered to the site of discomfort.

Those of skill in the art will recognize that a wide variety of delivery mechanisms are also suitable for the present invention.

The present brilaroxazine liposomes composition is useful in treating a mammal subject, such as humans, horses, and dogs. The present invention is particularly useful in treating humans.

The following examples further illustrate the present invention. These examples are intended merely to be illustrative of the present invention and are not to be construed as being limiting.

### EXAMPLES

### Example 1. Preparation of Brilaroxazine Liposomes

Table 1 shows the formulation composition of brilaroxazine liposomes.

TABLE 1

| No. | Ingredients list | Percentage | CAS No. |
|---|---|---|---|
| 1. | Brilaroxazine | 24.53 | 1239729-06-6 |
| 2. | Lecithin | 34.76 | 8002-43-5 |
| 3. | Cholesterol | 2.97 | 57-88-5 |
| 4. | Maltodextrin | 37.74 | 9050-36-6 |
| 5. | Purified water | Qs | NA |
| | (for lipid film | | |
| | hydration) | | |

Solvents used for lipid dry film preparation

| | | | |
|---|---|---|---|
| 6. | Chloroform | NA | 67-66-3 |
| | (dried out in | | |
| | the process) | | |
| 7. | Ethanol (dried | NA | 64-17-5 |
| | out in the | | |
| | process) | | |

The brilaroxazine liposomes were prepared by the lipid hydration method. Briefly, phosphatidylcholine and cholesterol were dissolved in a suitable solvent (chloroform and/or ethanol) and brilaroxazine was dissolved in the same solvent. Then this drug-lipid solution was dried at 45-50° C. in a rotary evaporator using the vacuum to remove the solvent completely. Once all the solvent was removed, a thin film formed in the round bottom flask. This round bottom flask containing lipid film was kept in a vacuum for 12-24 h to completely remove the traces of the solvents present in the thin lipid film. After 12-24 h of drying, the thin film was hydrated with 66 mL of 60° C. maltodextrin solution (concentration 39.57 mg/mL).

### Example 2. Particles Size and Zeta Potential Analysis of Brilaroxazine Liposomes

The prepared liposomes from Example 1 were viewed at different magnifications in an optical microscope for confirmation during the hydration process. Optical microscopic viewing at different magnifications (10×, 20×, and 40×) for confirmation of the prepared spherical-shaped liposomal vesicles occurred throughout the hydration process.

The liposomes were analyzed for particle size by DLS (dynamic light scattering) method for particle size analysis and drug content. The Z-average (particle size) was measured for the prepared liposomes. FIG. 2 shows particles size (Z-average) and particles distribution index in the liposomal dispersion by DLS measurement of brilaroxazine liposomes.

Zeta potentials was estimated from experimentally determined electrophoretic mobility of particles. The value of zeta potential indicates the stability of the colloidal dispersion.

Zeta potential (mV) value:
0 to 5—Rapid coagulation or flocculation
10 to 30—Incipient instability
30 to 40—Moderate stability
40 to 60—Good stability
>61—Excellent stability

In general, the colloidal dispersion with zeta potential values greater than positive 30 mV or less than negative 30 mV have high degrees of stability. The higher the zeta potential value (both positive and negative), the better the stability.

Table 2 shows particle size and zeta potential analysis of Liposomes by DLS method.

TABLE 2

| | | Zeta |
|---|---|---|
| | Particle Size | potential |
| Liposomes No. | (Z-Average) nm | (mV) |
| RP5063 | 630.9 | 38.8 |
| Liposomes | | |

### Example 3. Measurement of Drug Content by HPLC

Liposome drug content was analyzed by the HPLC method.

### HPLC System

Shimadzu HPLC system (LC-2030C Plus, Serial No: L21445711704 AE, Made in Japan), auto sampler, UV detector, data acquisition system. Equivalent system may be used as substitute.

### HPLC Column

Shimadzu Shim-Pack GIST C18, 5 μm, 250 ×4.6 mm column or equivalent.

Reagent preparation

Mobile Phase A
Dissolve 2.72 g KH2PO4 in 1000 mL of ultrapure water (0.02M Solution).
Adjust the pH to 3.0 with phosphoric acid.
Mix 90 parts of the above buffer with 10 parts of acetonitrile.
Filter through the membrane prior to use.

Mobile Phase B
Mix 90 parts of acetonitrile with 10 parts of ultrapure water. Adjust the pH to 3.0 with phosphoric acid.
Filter through the membrane prior to use.

Diluent
Prepare a mixture of acetonitrile and phosphate buffer (section 6.2.1.1.) (85:15) for drug content/encapsulation efficiency.

### Sample Preparation for Drug Content and Incarnation Efficiency

### Drug Content

Take the required amount of liposomes sample (whole dispersion) in a volumetric flask, add the volume of diluent needed (6.2.3), and mix well. Keep this mixture in a bath sonicator for 30-45 minutes at 60° C. Take the required volume of the prepared sample and dilute it to the required concentration. The assay/drug content concentration is 20 μg/mL.

### Incorporation Efficiency

Take the required amount of liposomes sample in a centrifuge tube and centrifuge the liposomes dispersion at 10000 rpm for 30 minutes at 20° C. Remove the supernatant solution and collect the pellet. Add the required diluent volume to the liposomal pellet, and mix well. Keep this mixture in a bath sonicator for 30-45 minutes at 60° C. Take the required volume of the prepared sample and dilute it to drug concentration of 20 μg/mL.

### Standard Solution

Prepare 20 μg/mL standard solution with the above diluent.

### Analysis

Set up the HPLC using the following parameter:

Column: Shimadzu Shim-Pack GIST C18, 5 μm, 250×4.6 mm column or equivalent

Flow rate: 1 mL/min

Injection volume: 20 μL

Detection: UV @215 nm

Column Temperature: 30° C.

Run time: 5-7 minutes

HPLC conditions: Mobile phase A, 25%; mobile phase B, 75%

### Identification

Compare the retention time of the standard/pure drug peak and sample drug peak

### Result

The HPLC chromatogram is shown in FIG. 3. The HPLC results show that drug content is 96% and drug incorporation efficiency into liposome is 73%.

The final formulation composition was consistent with brilaroxazine (24.53%), lecithin (34.75%), cholesterol (2.97%), maltodextrin (37.74%), and purified water for lipid film hydration.

### Example 4. Preparation of Liposomal Gel Formulations

The liposomal gel was prepared by incorporating a liposome dispersion in a gel formulation. First, the plain gel was prepared, and then the liposome dispersion was added and mixed thoroughly to result in the liposomal gel or lipogel. Various percentage of lipogel formulation (0.25% to 1.5% of brilaroxazine) was prepared according to need. Table 3 shows composition of the lipogel formulation.

TABLE 3

| Ingredients | Lipogel | |
|---|---|---|
| activity | Formulation - RPLG | Percentage (%) |
| Gelling agent | Carbomer 940 | 0.65-0.85 |
| Humectant | Glycerin | 5.00 |
| pH modifier | Triethanolamine | 0.10-0.25 |
| Preservative | Phenoxyethanol | 1.00 |
| Vehicle | Purified water | Qs |
| Brilaroxazine | RP5063 liposomal | Eq to 0.25% or 1.5% |
| | dispersion | of brilaroxazine |

The prepared liposomal gel formulation was evaluated for physical appearance and pH. All the gel formulations were viewed under an optical microscope for intact liposomes in the gel formulation. The liposomal gel formulation had white cream gel appearance, with pH 5-6, and microscope examination showed presence of liposomes. In addition, the liposome particles were intact in all gel formulations and stable.

### Example 5. Analysis of Lipogel by HPLC

Lipogel sample was placed in a volumetric flask and added with diluent and mixed well. Brilaroxazine content was analyzed by HPLC according to the same protocols of Example 3.

Comparing the retention time of the pure drug peak and sample peak, the drug content is calculated to be 95.12%.

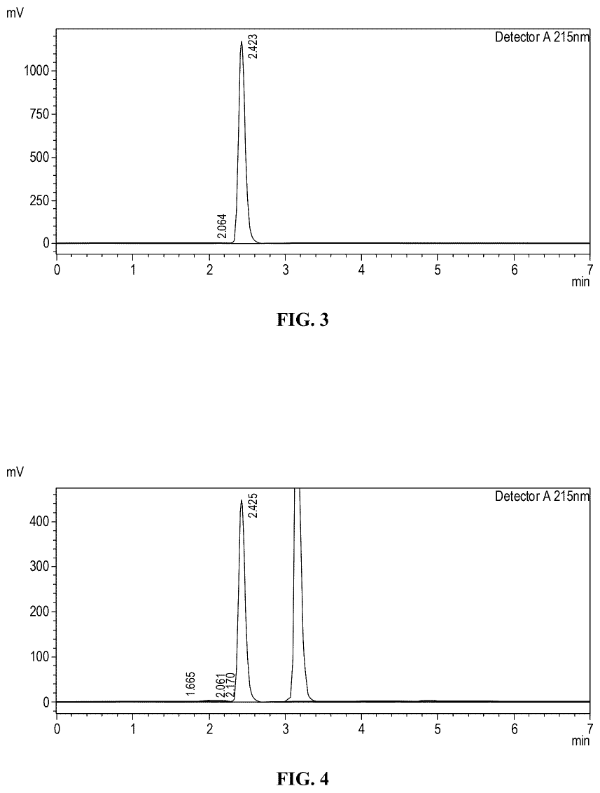

The HPLC chromatogram of a lipogel sample is shown in FIG. 4. The first peak is brilaroxazine and the second peak is excipient. Brilaroxazine peak is clear and separated from the excipient peak.

### Example 6. In Vitro Diffusion/Permeation Studies of Lipogel

The prepared lipogels were analyzed for drug diffusion/permeation using a Franz diffusion cell. The Franz diffusion cell was filled with pH 7.4 PBS buffer. The surface treated and pH 7.4 PBS neutralized regenerated cellulose dialysis membrane (MW cut off: 12000 to 14000) was placed on the receptor compartment, and a weighed amount of lipogel was placed in the donor compartment. The receptor solution was stirred with a magnetic stirrer, and the skin temperature was maintained in the diffusion cell by the circulation of temperature-controlled water in the outer jacket. At different time intervals, samples were withdrawn through a sample port and replaced with the same volume of plain PBS. The withdrawn sample was mixed with an equal volume of HPLC diluent and analyzed for drug content at different time intervals. The percentage of drug diffusion, flux rate, and permeation coefficient was calculated for each lipogel formulation. The time vs percentage drug diffusion/release was plotted using Graph Pad Prism Version 6.01 Software.

### HPLC Analysis of Drug Diffusion Samples

The HPLC analysis method, system, and columns were the same as described in Example 3 except a mixture of acetonitrile and phosphate buffer (60:40) was used for drug diffusion/permeation studies.

### Sample Preparation for Diffusion/Permeation Sample Analysis

Mix an equal volume of diluent with diffusion/permeation sample fluid taken from the Franz diffusion apparatus. Mix thoroughly in a vertex mixer and filter the solution through a syringe filter.

### Results

The results of HPLC chromatogram of the lipogel diffusion sample show a clear and separated brilaroxazine peak.

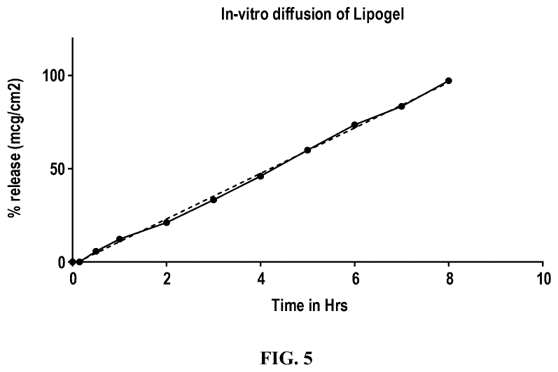

FIG. 5 shows in-vitro diffusion study graph of lipogel through the membrane. The release profile showed steady and sustained brilaroxazine release from the formulation throughout the study period of 8 hours.

Table 4 shows in vitro diffusion study results of Lipogel formulations.

TABLE 4

| | | Permeation | |
|---|---|---|---|
| | Flux | coefficient | R2 |
| Formulation | (μg/cm²/h) | (Cm/h) | Value |
| Lipogel | 12.02 | 3.28 | 0.9984 |
| Formulation | | | |
| With maltodextrin | | | |
| Lipogel | 5.48 | 1.92 | 0.9633 |
| Formulation | | | |
| Without | | | |
| maltodextrin | | | |

The lipogel formulation containing liposomes with maltodextrin show a better drug release profile and the higher flux and permeation value in the in vitro diffusion study than that of liposomes without maltodextrin. This may be due to the increased solubility of RP5063 in the liposomal gel and optimum particle size distribution in the formulation.

It is to be understood that the foregoing describes preferred embodiments of the present invention and that modifications may be made therein without departing from the scope of the present invention as set forth in the claims.

What is claimed is:

1. A liposome composition comprising bilayer lipid vesicles encapsulating an aqueous solution, wherein the bilayer lipid vesicles comprise one or more phospholipids, sterol, and brilaroxazine or a pharmaceutically acceptable salt thereof, and the aqueous solution comprises maltodextrin.

2. The liposome composition of claim 1, wherein the phospholipid comprises phosphatidylcholine, and sterol comprises cholesterol.

3. The liposome composition of claim 1, comprising 20-30% by weight of brilaroxazine.

4. The liposome composition of claim 1, comprising 30-45% by weight of maltodextrin.

5. The liposome composition of claim 1, wherein the average particle size of the lipid vesicles is between 500-750 nm.

6. The liposome composition of claim 1, comprising 20-30% by weight of brilaroxazine, and 30-45% by weight of maltodextrin.

7. The liposome composition of claim 6, wherein the average particle size of the lipid vesicles is between 500-750 nm.

8. A pharmaceutical composition comprising the liposome composition of claim 1, and a pharmaceutically acceptable carrier.

9. A gel formulation comprising the liposome composition of claim 1, a gelling agent and a humectant, wherein the gel formulation comprises brilaroxazine in an amount of 0.1-2% by weight, the formulation has a gel appearance, and the bilayer lipid vesicles are intact and stable in the gel.

10. The gel formulation of claim 9, wherein the gelling agent is carbomer 940, and the humectant is glycerin.

11. A gel formulation comprising the liposome composition of claim 7, a gelling agent and a humectant, wherein the gel formulation comprises brilaroxazine in an amount of 0.1-2% by weight, the formulation has a gel appearance, and the bilayer lipid vesicles are intact and stable in the gel.

12. The gel formulation of claim 11, wherein the gelling agent is carbomer 940, and the humectant is glycerin.
